(19) United States
(12) Patent Application Publication
(10) Pub. No.: US 2015/0102925 A1
(43) Pub. Date: Apr. 16, 2015

(54) SYSTEMS AND METHODS FOR DETECTION OF BIOLOGICAL CONDITIONS IN HUMANS

(71) Applicant: Aegis Industries, Inc., Rockville, MD (US)

(72) Inventors: Ivo Foldyna, Rockville, MD (US); Kenneth J. Stethem, Hailey, ID (US)

(21) Appl. No.: 14/302,037

(22) Filed: Jun. 11, 2014

Related U.S. Application Data
(60) Provisional application No. 61/833,785, filed on Jun. 11, 2013.

(51) Int. Cl. A61B 5/00; A61B 5/026; A61B 5/021; A61B 5/1455; A61F 7/00; A61B 5/08; A61B 3/11; A61M 5/172; A61N 1/39; A61B 5/0205; A61B 5/145

(52) U.S. Cl. CPC A61B 5/746; A61B 5/0205; A61B 5/026; A61B 5/021; A61B 5/14552; A61B 5/14532; A61B 5/08; A61B 3/11; A61M 5/1723; A61N 1/3987; A61F 7/00; A61B 5/0022
USPC 340/539.12; 600/301; 600/504; 600/485; 600/323; 600/365; 600/529; 600/558; 604/503; 607/6; 607/96

(57) ABSTRACT

A method detects a biological condition and detects a change in the biological condition of a mammal. The device determines if the change in the biological condition exceeds a predetermined threshold and sends a signal based at least in part on the determination.

### CROSS-REFERENCE TO RELATED APPLICATIONS

This application claims priority to and the benefit of U.S. Provisional Patent Application Ser. No. 61/833,785, entitled “METHOD AND APPARATUS FOR DETECTION OF SUDDEN DEATH IN HUMANS,” filed Jun. 11, 2013, the disclosure of which is hereby incorporated by reference herein in its entirety.

### INTRODUCTION

The present invention has applications in many fields, including (A) the purpose of summoning intervening help and (B) for purposes of permanent non-volatile recording of the exact time of the occurrence of sudden death.

Each year 359,400 people in the U.S. (about 1,000/day) experience EMS-assessed out-of-hospital non-traumatic Sudden Cardiac Arrest (SCA), and nine out of ten victims die. SCA is a sudden and unexpected pulse-less condition attributed to cessation of cardiac mechanical activity. It is usually caused by ventricular fibrillation, an abnormality in the heart's electrical system. When SCA occurs, blood stops flowing to the brain, the heart, and the rest of the body, and the person collapses. In fact, the victim is clinically dead and will remain so unless someone helps immediately.

On average, only 11.4% of EMS-treated non-traumatic SCA victims survive. Survival rates among young SCA victims are somewhat lower (8.6%). However, when victims are treated quickly, their chances of survival improve dramatically. If bystanders provide CPR and use an Automated External Defibrillator (AED) to treat the victim before EMS arrives, survival rates increase to 38%. In other words, lay bystanders who take action by calling 9-1-1, start CPR, and use the nearest AED, can mean the difference between life and death for victims of sudden cardiac arrest. For every minute without CPR and defibrillation, the victim's chance of survival decreases by 7-10%.

The American Heart Association statistical facts indicate that in the U.S., about 80 percent of all sudden cardiac arrests happen at home, and almost 40 percent are not witnessed. SCA victims can survive if they receive immediate CPR and are treated quickly with defibrillators. To be effective, this treatment must be delivered quickly—ideally, within three to five minutes after collapse.

Given this statistical information, approximately 730 Americans die from Sudden Cardiac Death at home each day. Tragically, 300 of such deaths occur not witnessed by anyone because the victim is alone or the potential witness is unaware of the victim's condition. For example, the victim and or the potential witness may be asleep. Survival can be as high as 90% if treatment is initiated within the first minutes after Sudden Cardiac Arrest.

There are many additional causes of precipitous death in humans due to but not limited to shock, aortic dissection, congestive heart failure, accompanied by hypoxia, polycystic disease of heart, familial endocardial fibroelastosis, Kawasaki's disease, anaphylaxis, ‘cafe coronary’, carbon monoxide, hydrogen sulfide, cyanide, nicotine, organophosphate pesticides, gastric rupture due to Mallory-Weiss syndrome, ulcers, septicemia, obstruction, bezoars, cerebrovascular lesions, and Sudden Infant Syndrome.

Generally, it is impossible to determine an exact time of death unless a trained medical professional is near the person who dies. Presently, and under the best circumstances, the exact time of death can be only estimated within a range of 20 minutes to 1 hour of the actual time of death.

The actual time of death is important in many situations. In criminal cases, an accurate estimation of the time of death can lead to discovering the identity of the assailant. It can eliminate some suspects while focusing attention on others. But the time of death is not confined to criminal investigations; it can also come into play in civil situations. Insurance payments may depend upon whether the insured individual was alive at the time the policy went into effect or if he died before the policy expired. Even a single day can be important. Likewise, property inheritance can hinge on when the deceased actually died.

### SUMMARY

The technologies disclosed herein include methods and devices for the non-invasive measurement of blood flow in humans, as well as monitoring of blood flow in humans. Certain embodiments detect sudden death in humans by means of a simple, user-wearable, and autonomous apparatus. The technologies disclosed herein indirectly detect sudden death in humans by monitoring the flow of blood in the circulatory system. Detection of stopped blood flow is an indication of death in humans. Upon such detection, notifications may be sent via: an audible alarm; a link to cellular telephone for automated emergency dispatch; a link to satellite communication system for automated emergency dispatch; and a link to Internet communication system for automated emergency dispatch.

Under supervised medical observation, many accepted methods exist to detect sudden death in humans. Such methods rely on monitoring the individual's blood pressure or rely on myriad of data that can are obtained by reading electrical signals produced by human body. Such methods of obtaining vital signs by monitoring the breathing, blood oxygen level, EKG/ECG, EEG and even blood pressure require the observed subject to be stationary. Portable, user-wearable and non-intrusive variants implementing such methods are not practical or reliable. As an example, during ventricular fibrillation, the heart continues to produce detectable electrical signals that require significant algorithmic processing to autonomously discriminate between this life threatening condition and normal heart beat. One generally accepted method of determining that a sudden death or condition such as the ventricular fibrillation has occurred is by observing the blood pressure drop to “zero”.

In one aspect, the technology relates to a method having: with a first device disposed on a mammal: detecting a biological condition; detecting a change in the biological condition; determining if the change in the biological condition exceeds a predetermined threshold; and sending a signal based at least in part on the determination. In an embodiment, the biological condition includes at least one of a blood flow, a blood pressure, a blood oxygen level, a blood sugar level, a respiration rate, a temperature, a perspiration, an electrical level, and a pupil dilation. In another embodiment, the signal includes at least one of an alarm signal, an initiation signal, and a communication signal. In yet another embodiment, the signal includes an alarm signal and the method further includes, emitting, from the first device disposed on the mammal, at least one of an audible and visual alarm. In still another embodiment, the signal includes an initiation signal and the method further includes initiating a treatment to the mammal.

In another embodiment of the above aspect, the treatment includes administering to the mammal at least one of a medicament, an electric shock, and a temperature stimulation. In an embodiment, the treatment is administered by a second device in communication with the first device. In another embodiment, the signal includes a communication signal, wherein the communication signal is sent to a second device disposed remote from the first device. In yet another embodiment, at least one of the detecting operations is performed without penetrating a skin surface of the mammal.

In another aspect, the technology relates to a method having a device disposed on a mammal: detecting a change in a biological condition, the change indicative of a death of the mammal; and emitting an alarm signal. In an embodiment, the alarm signal includes at least one of an audible signal, a visual signal, and a communication signal. In another embodiment, the method further includes storing, on the first device, information regarding at least one of the mammal, a geographic location of the mammal, the biological condition, and a time of day. In yet another embodiment, the method further includes sending the information from the first device to a remote database. In still another embodiment, the method further includes detecting, with the first device, an environmental condition external to the mammal.

In another aspect, the technology relates to a system having a wearable device having: a housing; an attachment element secured to the housing; a sensor for sensing a biological condition of a mammal; an emitter; a processor disposed in the housing and in communication with the sensor and the emitter; and a storage device in communication with the processor for storing information regarding the biological condition. In an embodiment, the emitter includes at least one of a speaker and a light-emitting element. In another embodiment, the system further includes a transmitter in communication with the processor for transmitting a signal to a remote device. In yet another embodiment, the remote device includes at least one of an emergency dispatch system, a medical monitoring station, and a medical device. In still another embodiment, the sensor is disposed proximate a skin surface of the mammal. In yet another embodiment, the sensor has a transcutaneous blood pressure sensor.

### BRIEF DESCRIPTION OF THE DRAWINGS

FIGS. 1A and 1B depict embodiments of a system for detecting a biological condition in a mammal.

FIG. 2 depicts an embodiment of a monitoring device.

FIG. 3 depicts a method of monitoring a biological condition of a mammal.

FIG. 4 depicts a thermal image of a living human with normal blood flow.

FIG. 5 depicts a thermal image of a human arm with restricted blood flow, simulating conditions when the flow of blood ceases in a human body.

FIG. 6 depicts an embodiment of a monitoring device.

FIGS. 7 and 8 depict other embodiments of a monitoring device.

FIG. 9 illustrates one example of a suitable operating environment in which one or more of the aspects of the disclosure may be implemented.

FIG. 10 depicts a network in which the various systems and methods disclosed herein may operate.

### DETAILED DESCRIPTION

All illustrations of the drawings are for the purpose of describing selected embodiments in accordance with the present technology and are not intended to limit the scope of the present technology.

The technologies described herein may be utilized to monitor the biological conditions of a mammal, such as a human. The biological conditions include, but are not limited to blood flow, blood pressure, blood oxygen level, blood sugar level, respiration rate, temperature, perspiration, electrical activity level (e.g., brain wave activity), and pupil dilation. Any of these conditions may alter or change when the human is subject to environmental or physical stimuli. For example, a gunshot victim may experience a precipitous loss in blood pressure, or a diabetic may begin to experience low blood sugar. Respiration rate, body temperature, and perspiration may increase when the human is subject to exercise or other stress. In certain circumstances, certain of these biological conditions may be indicative of shock, death, heart attack, stroke, etc. In any case, information about the biological condition may be sent from the monitoring device to a remote caregiver, law enforcement, coroner, or other entity desiring the information. In the case of a remote caregiver, for example, a companion or nurse may be notified of the need for medical intervention on behalf of the wearer of the monitoring device. Law enforcement officials may wish to determine the exact time and condition of death. In other embodiments, the biological condition information may be stored on the monitoring device until retrieved.

In certain embodiments, the biological condition may be detected or sensed by a sensor that is either external to or internal to the patient. For example, an implanted or external temperature sensor may detect elevated body temperature that may be indicative of stress or sickness. A blood sugar detector can detect blood sugar levels. Blood pressure sensors and pulse oxymeters can detect conditions relative to the blood. In embodiments, the monitoring device can communicate with a medical device disposed on the wearer. For example, the detected low blood sugar levels can be initiate delivery of insulin from an insulin infuser also worn by the wearer. In other embodiments, the monitoring device may be incorporated into such a medical device. Other embodiments and applications will be apparent to a person of skill in the art.

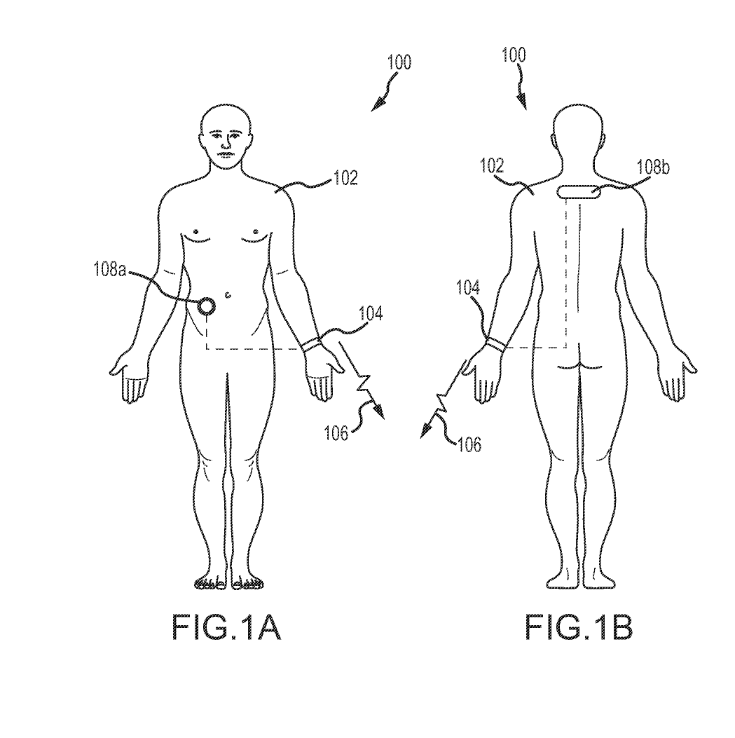

FIGS. 1A and 1B depict a embodiments of a system 100 for detecting a biological condition in a mammal 102, such as a human. The system 100 includes a monitoring device 104, in this case, having a form factor similar to a wristwatch. In other embodiments, the monitoring device 104 may be worn as a ring, an earring, belt, or may be secured directly to the skin (e.g., in a discrete location) with a biocompatible adhesive. The monitoring device 104 is described in further detail below. In general, however, the monitoring device 104 includes one or more sensors, processors, or other components that allow for monitoring of biological conditions, storage of information, generation of alarms and/or transmission signals 106, etc. The system 100 may also include a remote medical device 108a, 108b, such as an insulin infuser, wearable automated external defibrillator (AED), temperature augmentation device. In the depicted FIG. 1A, an insulin infuser 108a may be in communication with the monitoring device for delivering insulin to the human 102 in the event of detected low blood sugar. In another embodiment, depicted in FIG. 1B, a convective cooling device 108b may be disposed on the upper back or neck of the human 102 and activated when elevated body temperatures are detected. Such an embodiment may be particularly useful for endurance athletes to keep cool and improve performance.

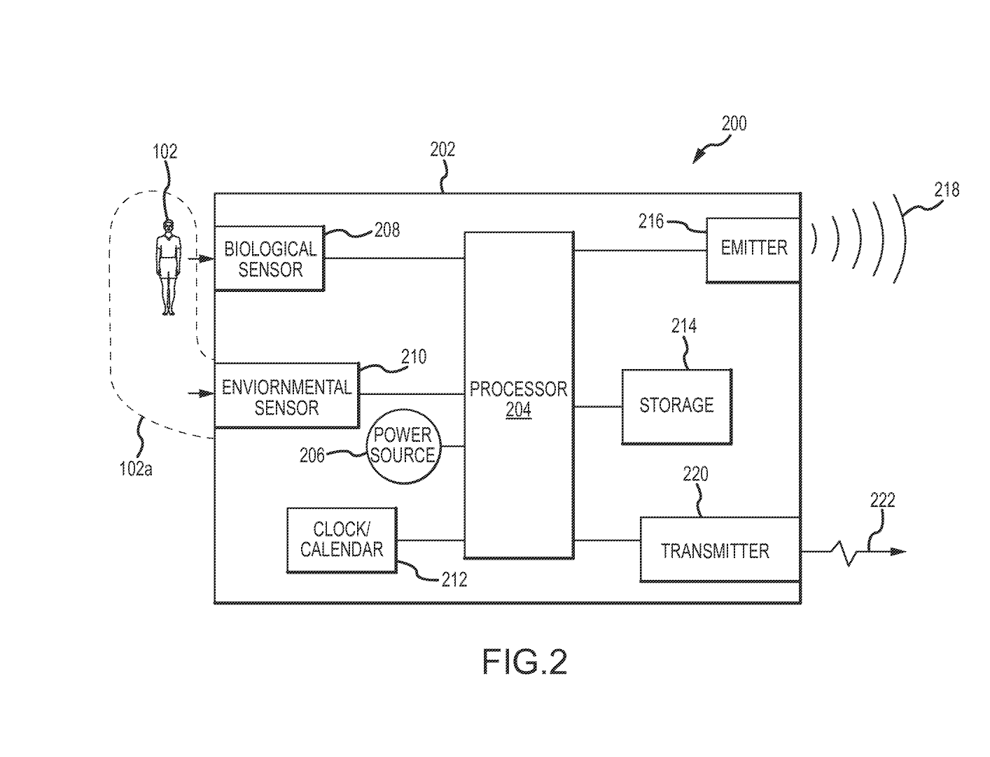

FIG. 2 depicts an embodiment of a monitoring device 200. The device 200 includes a housing 202 in which are disposed a number of components. A processor 204 communicates with the various components, processes signals, emits alerts, controls transmission signals, etc. A power source 206 such as a battery provides power. The monitoring device can include one or more sensors. In the depicted embodiment, a biological sensor 208 detects biological conditions of the human 102 wearing the monitoring device 200. Such biological conditions are described herein. Additionally, an environmental sensor 210 may detect conditions within the environment 102a in which the human 102 is located. Such an environmental sensor 210 may be useful to enable a person such as a law enforcement officer to determine the conditions at the time of, for example, a wearer's death. Such a sensor 210 can include a temperature or barometric pressure sensor, location sensor (e.g., a global positioning (GPS) sensor or signal emitter which may be detected by a remote device), or even a microphone or video receiver. Sound and/or video recorded by such a sensor 210 may aid law enforcement in determining the circumstances surrounding the wearer's death. A clock or calendar 212 may be discrete from or incorporated into the processor 204. A data storage element 214 is used to store data regarding biological or environmental conditions, time and date, etc. An emitter 216 can be used to generate an audio or visual alarm 218 which can alert those nearby to the need of the human 102 for aid or medical or other intervention. A transmitter 220 can send signals 222 to a remote device such as a medical device (described above), a database, a medical monitoring system, or an emergency dispatch system.

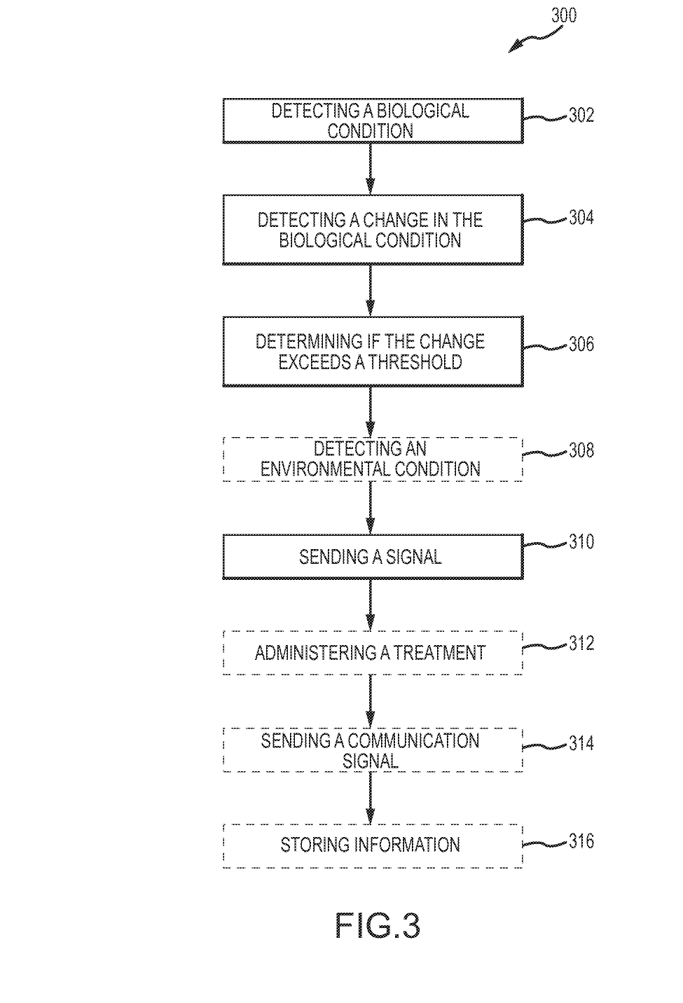

FIG. 3 depicts a method 300 of monitoring a biological condition of a mammal. The method begins with detecting a biological condition of the mammal, operation 302. As described above, the biological condition may be at least one of a blood flow, a blood pressure, a blood oxygen level, a blood sugar level, a respiration rate, a temperature, a perspiration, an electrical level, and a pupil dilation. The detected biological condition may be constantly monitored, or monitored intermittently until a change in the biological condition is detected, operation 304. The change may then be compared to a predetermined threshold, operation 306, to determine if a threshold is exceeded. The method 300 also can include detecting an environmental condition, operation 308. This operation may occur on an ongoing basis or intermittently.

If it is determined that the change in biological condition exceeds the threshold, the monitoring device may send a signal in operation 310. The signal may include on or more types of signals. For example, the signal may be an alarm signal, an initiation signal, or a communication signal. An alarm signal may be either or both of an audible or visual alarm. An initiation signal initiates a treatment to the wearer, operation 312. A treatment may include, for example, an infusion of a medicament, as described generally above. In other embodiments, the treatment may include the administration of an electric shock (e.g., by a body-worn AED), or a temperature stimulation (e.g., activation of the convective cooling module described above). Of course, such treatments may be administered by the monitoring device itself, or a device disposed remotely therefrom. A communication signal may also be sent from the device, operation 314. The communication signal may be sent to a device remote from the monitoring device. Such remote devices may include an emergency dispatch system, a medical monitoring station (e.g., a nurse's station), a remote database for storage and analysis, and a medical device. The communication signal may include information regarding the wearer of the monitoring device, a geographic location of the wearer, the biological condition, and a time of day.

### Example 1

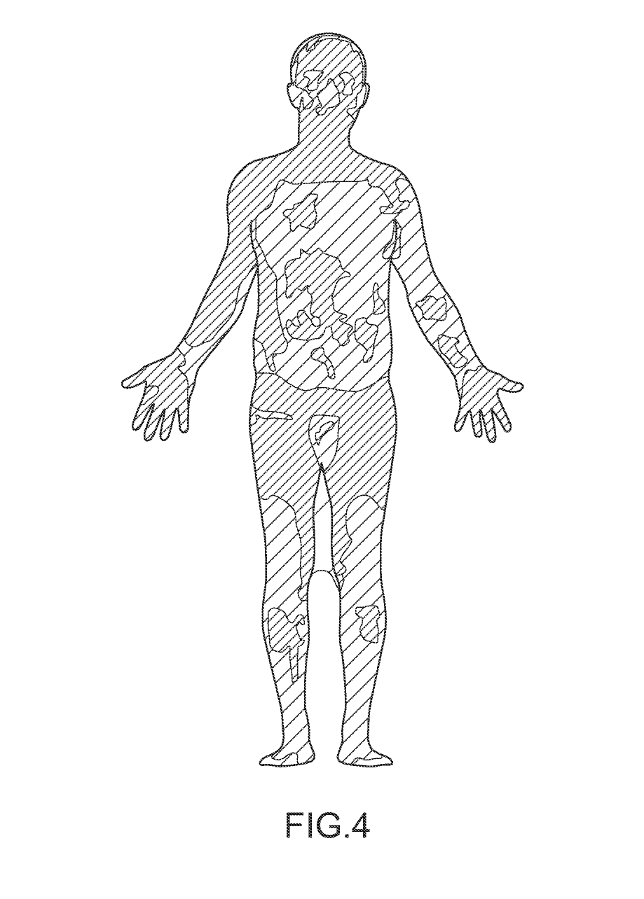

The technologies described above can be utilized in a system and method that detects death in humans. Such devices and methods are described with regard to Example 1, in FIGS. 4-9, below. In general, a blood flow monitor is provided for a person to wear, monitoring or sampling the flow of blood at a body location adjacent to the monitor. If the monitor detects an absence of blood flow, it implies that the wearer of the monitor has died. The monitor then records information about the wearer's death that may be beneficial for others. The present technology provides a simple, reliable, highly portable, low power, and eco-friendly apparatus along with a method that is inexpensive to implement. The present technology has numerous benefits and applications. An embedded 64-bit registration number allows separate apparatuses of the present technology to be traceable, since no two registration numbers will be the same.

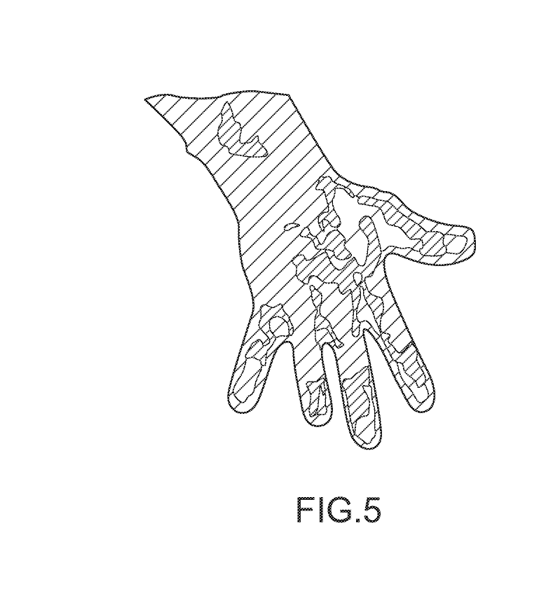

FIG. 4 depicts a thermal image of a living human with normal blood flow. The shading gradient within the image represents the profound variance of temperature on the surface of the body. FIG. 5 depicts a thermal image of a human arm with restricted blood flow, simulating conditions when the flow of blood ceases in a human body. As the blood flow stops the lack of warm, oxygen rich arterial blood supply causes immediate temperature drop and the body surface temperature variance diminishes. The presently-described embodiment monitors blood flow, such as in FIG. 4. If a change in blood flow, in this case, an absence thereof, as seen in FIG. 5, subsequent operations are performed by the device. These include creating an alert, storing death related data, and sending notifications.

The depicted embodiment provides a method for detecting death, as well as an apparatus the monitor which utilizes the method. The apparatus monitors and detects anatomical and physiological differences between presence and absence of blood flow in the human body. The monitoring device is non-invasive and worn by a potential “victim” (i.e., one who might succumb to sudden death). The exact form, shape, and placement of the apparatus depends on the specific sensor used to detect the flow of blood.

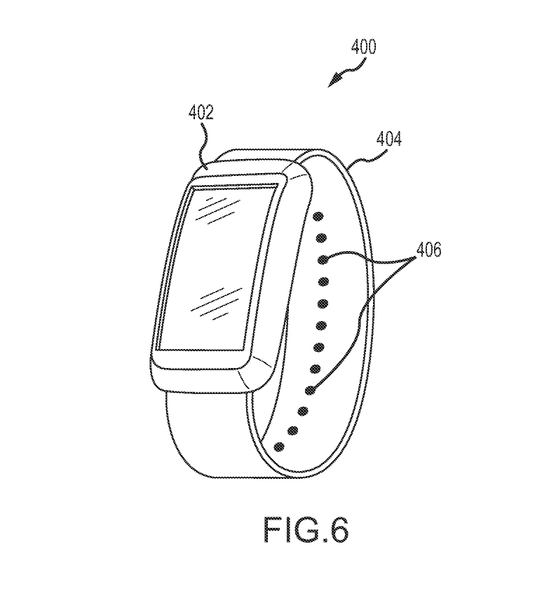

FIG. 6 depicts an apparatus 400 worn on the wrist equipped with multiple temperature detectors, an on-board microprocessor, and a power source. The blood monitoring apparatus comprises a main housing 402, an attachment element 404 such as a wrist band, a microprocessor chipset, a plurality of blood flow detectors 406, a low power transmitter, a data storage device, an alarm, and a power source. The blood flow detectors 406 act as a sensing system while the low power transmitter and the data storage device act as a reporting elements. The combination of the sensing system and reporting elements allows for access to the monitoring of blood flow in the human circulatory system. The main housing 402 encloses most of the components of the apparatus: a microprocessor, a transmitter, a data storage device (e.g., non-volatile memory), an alarm, and a power source. The attachment element 404 connects to the main housing 402 and allows the apparatus 400 to be secured to a wearer. The various electronic components, described in more detail above, are controlled by the microprocessor, with the blood flow detector, the low power transmitter, the data storage device, and the alarm being electronically connected to the microprocessor. The power source is electrically connected via the microprocessor to the blood flow detector, the transmitter, the data storage device, and the alarm, supplying the necessary energy for the apparatus 400 to operate. The apparatus 400 is secured to a user by the attachment element 404, with the main housing 402 being pressed against the wearer's body. The apparatus 400 is designed to be used in conjunction with the methods described herein. The apparatus 400 utilizes a blood flow detector to detect blood flow (or lack thereof, indicating death), record data after detecting a death, and sending out notifications in the event of a death.

In certain embodiments, the blood flow detector and the microprocessor can be separated. For example, the blood flow detector could be integrated into a watch band, ring, earring, or other accessory while the microprocessor chipset is separately housed in a fob or similar utilitarian apparatus. The microprocessor would then interrogate the blood flow detector for data using, for example, radio frequency identification (RFID) or other wireless or wired connections. In this scenario, a secondary power source would be provided for the blood flow detector, since it would be separated from the power source associated with the microprocessor. Additionally, the data storage device can accessed utilizing RFID technology. This allows stored data to be remotely retrieved and read, which enables a wider range of applications of the present invention.

In the depicted embodiment, the blood flow detector utilizes microelectromechanical systems (MEMS) sensors 406, while the microprocessor may be from the Texas Instruments MPS430 family. A real-time clock with integrated crystals may be provided by Maxim Integrated, while the transmitter may be from the BLE-Stack 1.3 low energy family produced by Texas Instruments. Cell phones and satellite communication devices to be used with the present technology can be provided by numerous companies, such as the iPhone from Apple or a satellite communication device from Iridium Communications, DeLorme, or Spot Inc.

The blood monitoring apparatus 400 can be integrated into various accessories, with said accessories acting as the main housing. For example, the blood monitoring apparatus could be integrated into a wristwatch (such as shown in FIG. 6), a bracelet, a pendant, a ring, an earring, a headband, a pair of glasses, or a non-contact detector or detector integrated in clothing. In some of the above embodiments, specifically the wristwatch, the attachment element 404 takes the form of a flexible band and buckle that wraps around a user's wrist. In other embodiments, such as the bracelet and the ring, the attachment means and the main housing are the same, with an annular body securing the apparatus around a user's wrist, arm, or finger, in addition to housing the components of the apparatus.

The blood flow detectors 406 include miniature temperature sensors placed alongside the watch band, which are used by the microprocessor to detect the onset of Algor mortis. In this specific application, during regular blood flow the temperature will exhibit a great variance, which is monitored by the microprocessor. However, if blood flow stops, the monitored temperature begins to approach an equilibrium. This change in temperature as measured along the wrist is interpreted by the microprocessor as an alarm condition for sudden death.

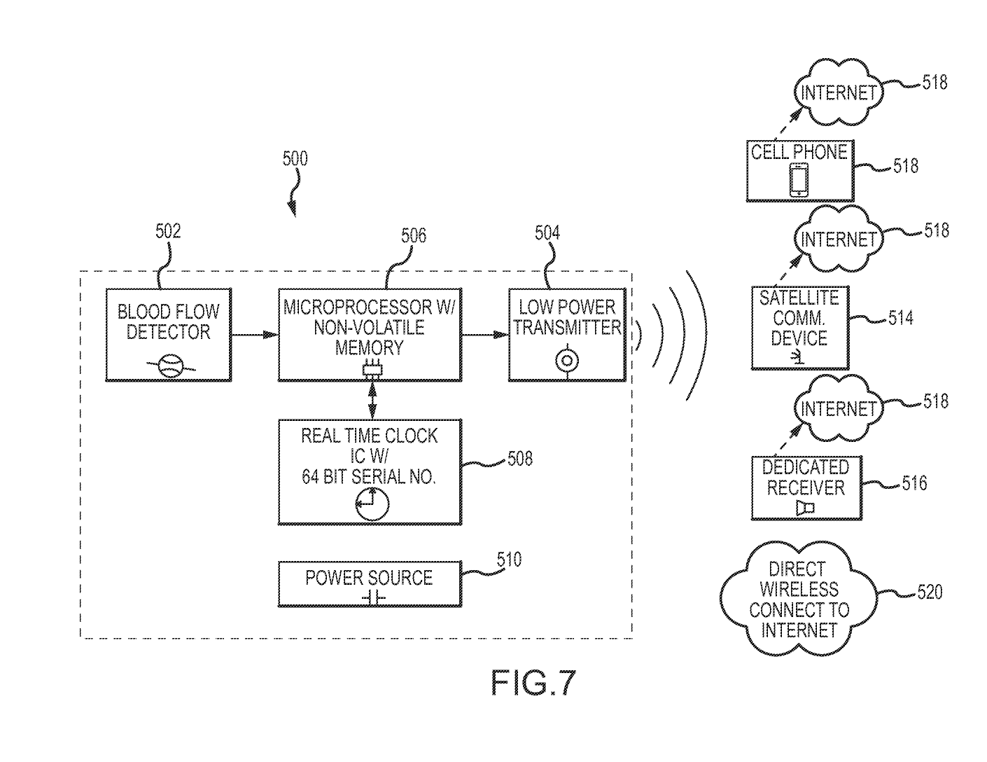

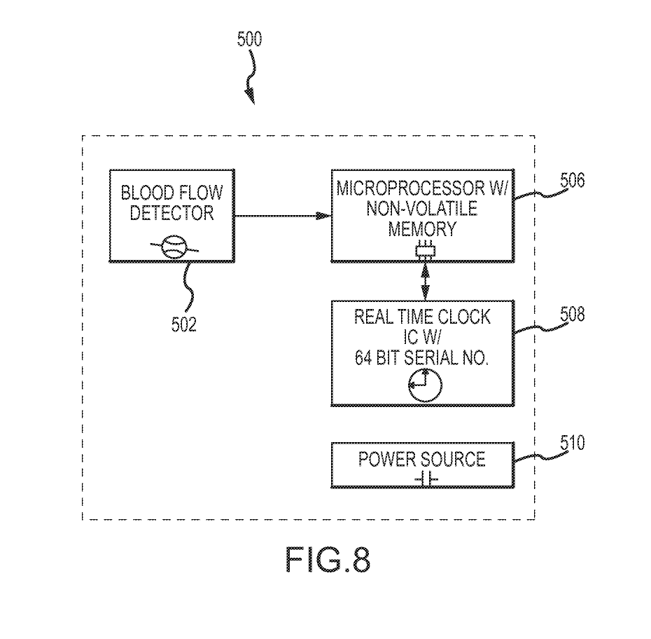

FIGS. 7 and 8 depict other embodiments of a monitoring device. FIG. 7 depicts a monitoring device 500 with an integrated blood flow detector 502 and an integrated Bluetooth transmitter 504. A microprocessor and memory module 506 communicates with a clock 508. A power source 510 provides power. The transmitter 504 can communicate with a smart phone or laptop 512, a satellite communication device 514, or a dedicated receiver 516 such as a nurse's station. Any of these devices may then connect to the internet 518. In an alternative embodiment, the transmitter may communicate directly with the internet 520

FIG. 8 depicts a “standalone” monitoring device 500 with an integrated blood flow detector 502 and a microprocessor and memory module 506 in communication with a clock 608. A power source 510 provides power. This monitoring device 500 is “standalone” assembly for the purpose of permanent non-volatile recording of the exact time of the occurrence of sudden death, but does not include a transmitter and other components depicted in FIG. 7.

The method utilized with the devices described in Example 1 monitors blood flow, determines whether blood flow is present or absent, indicates death in the absence of blood flow, records post-mortem data, and sends out post-mortem indications. Monitoring blood flow is a continuous or a sample based and non-invasive process, and may be carried out using a variety of sub-steps. In the present embodiment, the sub-steps for monitoring blood flow specifically monitor blood pressure, as blood pressure is required for blood flow; therefore, if no blood pressure is detected it can be inferred that blood flow has stopped. In other words, death results in a cessation of blood flow, which subsequently results in blood pressure dropping to “zero”. The blood flow monitor can utilize several different methods, which include but are not limited to the following: arterial and venous blood temperature difference detection; blood flow sensing by the thermo-transfer (calorimetric) principle; blood flow sensing by measuring the change in capacitance of the human body; blood flow sensing by measuring the change in inductance of the human body; blood flow sensing by measuring the change of oxygen level in arterial blood; blood flow sensing by measuring the change in sound waves produced by the human body; blood flow sensing by measuring the change in the electromagnetic radiation or field produced by the human body; blood flow sensing by measuring the change in the thermo-magnetic radiation or field produced by the human body; and blood flow sensing obtained by a sensor employing nanotechnology, which may be located within the human body or placed on the body surface. Other methods that provide continuous monitoring of blood flow may be used, such that death can be declared when there is an absence of blood flow.

When death is detected (e.g., blood flow is no longer detected) the monitoring device records death related data to a data storage device. The data comprises a time of death, as indicated by the real time clock. Additional information may also be included, provided the necessary components, such as recording position coordinates utilizing a GPS module. In addition to recording death related data, notifications are sent out utilizing the low power transmitter. These notifications can be sent to cell phones, satellite communication devices, dedicated receivers, or any combination thereof, and subsequently routed through the internet. Potentially, the low power transmitter may utilize a direct wireless connection to the internet, bypassing the intermediate steps.

The notifications can be sent to numerous sources, depending on the usage of the present invention. For example, in a home setting notifications might be sent to emergency service personnel and family members of the wearer. By notifying emergency service personnel the present invention may reduce response times between death and treatment, subsequently helping to save a person's life. This is because though stoppage of blood flow is clinically defined as death, persons who suffer these symptoms (such as with a sudden cardiac arrest) may still be revived if treatment is provided rapidly. The notifications sent to family members allow the family members to act as first responders, giving basic treatment to a person whose blood flow has stopped. In addition to or in place of notifications sent to family members, an alarm can be activated when blood flow is stopped. The alarm is ideally audible or visual, which have the best chances of alerting persons who may be asleep or otherwise distracted. In addition to notifying others that a person has clinically died and is in need of medical attention, the present invention provides peace of mind for loved ones of a user. A spouse who may have difficulty sleeping due to concern for the well-being of a loved one will ideally be comforted by knowing the present invention will alarm them in the event of an incident, and thus improve their quality of sleep. In other embodiments utilized by the military, police, or emergency responders, notifications may be sent out to squad mates. For example, an entire squad may be equipped with blood monitoring apparatuses, allowing squad mates to instantly be alerted when other troops or officers have be killed.

The death related data recorded by the present technology is useful in several different fields, such as forensics, religion, insurance, and legal. More specifically, the time of death holds importance in various matters. Some religions place importance upon the time of death. In forensics, the time of death is an important datum in and of itself. For insurance and legal purposes, the time of death may be important in determining whether policies or legal agreements had expired or not. Time of death may also settle inheritance issues, especially when multiple people with related or shared possessions die around the same time.

Many useful applications can be achieved by extrapolating the technologies described herein. Paraphrasing above the above, one such example is summoning intervening help in an instance of sudden death when the victim is alone or the sudden death conditions is not obvious. As a user wearable, non-intrusive apparatus, the present invention can monitor and detect anatomical and physiological differences when blood flow ceases, at which point an alert is triggered and help is summoned. A second example is the provision of permanent non-volatile recording of the exact time of death in the case of sudden death. As a user wearable, non-intrusive apparatus the present invention can monitor and detect anatomical and physiological differences when blood flow ceases, recording the exact time of such an event. The time of death, as stored data, may then be retrieved at a later time.

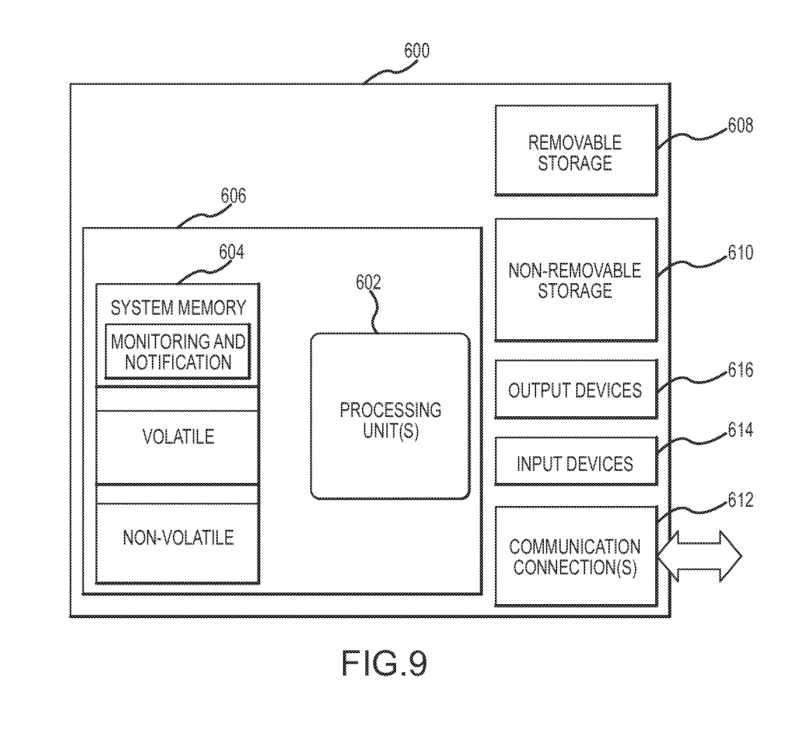

Having described various exemplary methods to perform the biological condition monitoring, the disclosure will now describe systems that may be employed to perform the methods disclosed herein. FIG. 9 and the additional discussion in the present disclosure are intended to provide a brief general description of a suitable computing environment in which the disclosed embodiments and/or portions thereof may be implemented. Although not required, the embodiments described herein may be implemented as computer-executable instructions, such as by program modules, being executed by a computer, such as a client workstation or a server, including a server operating in a cloud environment. Generally, program modules include routines, programs, objects, components, data structures and the like that perform particular tasks or implement particular abstract data types. Moreover, it should be appreciated that the disclosed embodiments and/or portions thereof may be practiced with other computer system configurations, including hand-held devices, multi-processor systems, microprocessor-based or programmable consumer electronics, network PCs, minicomputers, mainframe computers and the like. The disclosed embodiments may also be practiced in distributed computing environments where tasks are performed by remote processing devices that are linked through a communications network. In a distributed computing environment, program modules may be located in both local and remote memory storage devices.

FIG. 9 illustrates one example of a suitable operating environment 600 in which one or more of the present embodiments may be implemented. This is only one example of a suitable operating environment and is not intended to suggest any limitation as to the scope of use or functionality. Other well-known computing systems, environments, and/or configurations that may be suitable for use include, but are not limited to, personal computers, server computers, hand-held or laptop devices, multiprocessor systems, microprocessor-based systems, programmable consumer electronics such as smartphones, network PCs, minicomputers, mainframe computers, distributed computing environments that include any of the above systems or devices, and the like.

In its most basic configuration, operating environment 600 typically includes at least one processing unit(s) 602 and memory 604. Depending on the exact configuration and type of computing device, memory 604 (instructions to perform secure compression and/or secure decryption) may be volatile (such as RAM), non-volatile (such as ROM, flash memory, etc.), or some combination of the two. Memory 604 may store computer instructions related to performing the monitoring and notification functions described herein. Memory 604 may also store computer-executable instructions that may be executed by the processing unit(s) 602 to perform the methods disclosed herein.

This most basic configuration is illustrated in FIG. 9 by dashed line 606. Further, environment 600 may also include storage devices (removable, 608, and/or non-removable, 610) including, but not limited to, magnetic or optical disks or tape. Similarly, environment 600 may also have input device(s) 614 such as keyboard, mouse, pen, voice input, etc. and/or output device(s) 616 such as a display, speakers, printer, etc. Also included in the environment may be one or more communication connections, 612, such as an Ethernet adaptor, a modem, a Bluetooth adaptor, WiFi adaptor, etc.

Operating environment 600 typically includes at least some form of computer readable media. Computer readable media can be any available media that can be accessed by processing unit(s) 602 or other devices comprising the operating environment. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other tangible medium which can be used to store the desired information. Communication media embodies computer readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The operating environment 600 may be a single computer operating in a networked environment using logical connections to one or more remote computers. The remote computer may be a personal computer, a server, a router, a network PC, a peer device or other common network node, and typically includes many or all of the elements described above as well as others not so mentioned. The logical connections may include any method supported by available communications media. Such networking environments are commonplace in offices, enterprise-wide computer networks, intranets and the Internet.

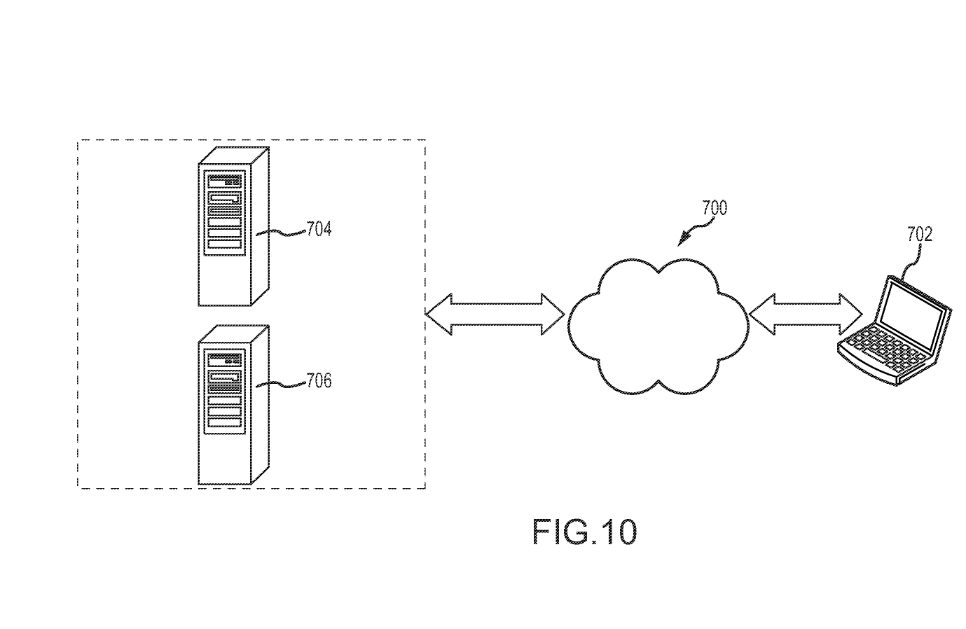

FIG. 10 is an embodiment of a network 700 in which the various systems and methods disclosed herein may operate. In embodiments, a client device, such as client device 702, may communicate with one or more servers, such as servers 704 and 706, via a network 708. In embodiments, a client device may be a laptop, a personal computer, a smart phone, a PDA, a netbook, or any other type of computing device, such as the monitoring devices depicted herein. In embodiments, servers 704 and 706 may be any type of device, such as the monitoring and notification devices described herein. Network 708 may be any type of network capable of facilitating communications between the client device and one or more servers 704 and 706. Examples of such networks include, but are not limited to, LANs, WANs, cellular networks, and/or the Internet.

In embodiments, the various systems and methods disclosed herein may be performed by one or more server devices. For example, in one embodiment, a single server, such as server 704 may be employed to perform the systems and methods disclosed herein, such a performing a primitive secure compression operation on data. Client device 702 may interact with server 704 via network 708 in order to request, view, operate upon, or otherwise access the raw or secure compressed data disclosed herein, etc., or any other object, property, and/or functionality disclosed herein. In further embodiments, the client device 706 may also perform functionality disclosed herein.

In alternate embodiments, the methods and systems disclosed herein may be performed using a distributed computing network, or a cloud network. In such embodiments, the methods and systems disclosed herein may be performed by two or more servers, such as servers 704 and 706. Although a particular network embodiment is disclosed herein, one of skill in the art will appreciate that the systems and methods disclosed herein may be performed using other types of networks and/or network configurations.

The aspects of the disclosure described herein may be employed using software, hardware, or a combination of software and hardware to implement and perform the systems and methods disclosed herein. Although specific devices have been recited throughout the disclosure as performing specific functions, one of skill in the art will appreciate that these devices are provided for illustrative purposes, and other devices can be employed to perform the functionality disclosed herein without departing from the scope of the disclosure.

This disclosure described some embodiments of the present technology with reference to the accompanying drawings, in which only some of the possible embodiments were shown. Other aspects can, however, be embodied in many different forms and should not be construed as limited to the embodiments set forth herein. Rather, these embodiments were provided so that this disclosure was thorough and complete and fully conveyed the scope of the possible embodiments to those skilled in the art.

Although specific embodiments were described herein, the scope of the technology is not limited to those specific embodiments. One skilled in the art will recognize other embodiments or improvements that are within the scope and spirit of the present technology. Therefore, the specific structure, acts, or media are disclosed only as illustrative embodiments. The scope of the technology is defined by the following claims and any equivalents therein.

What is claimed is:

1. A method comprising:
with a first device disposed on a mammal:
detecting a biological condition;
detecting a change in the biological condition;
determining if the change in the biological condition exceeds a predetermined threshold; and
sending a signal based at least in part on the determination.

2. The method of claim 1, wherein the biological condition comprises at least one of a blood flow, a blood pressure, a blood oxygen level, a blood sugar level, a respiration rate, a temperature, a perspiration, an electrical level, and a pupil dilation.

3. The method of claim 1, wherein the signal comprises at least one of an alarm signal, an initiation signal, and a communication signal.

4. The method of claim 3, wherein the signal comprises an alarm signal and the method further comprises, emitting, from the first device disposed on the mammal, at least one of an audible and visual alarm.

5. The method of claim 3, wherein the signal comprises an initiation signal and the method further comprises initiating a treatment to the mammal.

6. The method of claim 5, wherein the treatment comprises administering to the mammal at least one of a medicament, an electric shock, and a temperature stimulation.

7. The method of claim 5, wherein the treatment is administered by a second device in communication with the first device.

8. The method of claim 3, wherein the signal comprises a communication signal, wherein the communication signal is sent to a second device disposed remote from the first device.

9. The method of claim 1, wherein at least one of the detecting operations is performed without penetrating a skin surface of the mammal.

10. A method comprising:
with a device disposed on a mammal:
detecting a change in a biological condition, the change indicative of a death of the mammal; and
emitting an alarm signal.

11. The method of claim 10, wherein the alarm signal comprises at least one of an audible signal, a visual signal, and a communication signal.

12. The method of claim 10, further comprising storing, on the first device, information regarding at least one of the mammal, a geographic location of the mammal, the biological condition, and a time of day.

13. The method of claim 12, further comprising sending the information from the first device to a remote database.

14. The method of claim 10, further comprising detecting, with the first device, an environmental condition external to the mammal.

15. A system comprising:
a wearable device comprising:
a housing;
an attachment element secured to the housing;
a sensor for sensing a biological condition of a mammal;
an emitter;
a processor disposed in the housing and in communication with the sensor and the emitter; and
a storage device in communication with the processor for storing information regarding the biological condition.

16. The system of claim 15, wherein the emitter comprises at least one of a speaker and a light-emitting element.

17. The system of claim 15, further comprising a transmitter in communication with the processor for transmitting a signal to a remote device.

18. The system of claim 17, wherein the remote device comprises at least one of an emergency dispatch system, a medical monitoring station, and a medical device.

19. The system of claim 15, wherein the sensor is disposed proximate a skin surface of the mammal.

20. The system of claim 19, wherein the sensor comprises a transcutaneous blood pressure sensor.
